(19) United States
(12) Patent Application Publication
(10) Pub. No.: US 2026/0199530 A1
(43) Pub. Date: Jul. 16, 2026

(54) KIM-1 ACTIVE TARGETING GOLD NANOPARTICLES, PREPARATION METHOD AND APPLICATION THEREOF

(71) Applicant: Guangzhou First People's Hospital, Guangzhou (CN)

(72) Inventors: Bujie DU, Guangzhou (CN); Yuming QI, Guangzhou (CN); Xingya JIANG, Guangzhou (CN)

(73) Assignee: Guangzhou First People's Hospital, Guangzhou (CN)

(21) Appl. No.: 19/566,957

(22) Filed: Mar. 13, 2026

(30) Foreign Application Priority Data
Oct. 24, 2025 (CN) 202511526448.1

(51) Int. Cl. A61K 49/04; A61K 33/38; A61K 38/06; A61K 38/48; B82Y 5/00

(52) U.S. Cl. CPC A61K 49/0428; A61K 33/38; A61K 38/063; A61K 38/482; B82Y 5/00

(57) ABSTRACT

The invention discloses KIM-1 active targeting gold nanoparticles, a preparation method, and an application thereof, which belongs to the technical field of medical materials. The gold nanoparticles are modified on the surface of the gold nanoparticles with serine as a targeting ligand, which has high affinity and specificity for the kidney injury molecule KIM1. The preparation of GS-AuNPs is first synthesized, and then it is dissolved in PBS. After pH adjustment, EDC and NHS are added, serine is added, the pH is adjusted, and the product is obtained by ultrafiltration and centrifugation. The particles can be used as a CT contrast agent to detect KIM-1 in the kidney, and can also cooperate with phosphatidylcholine-modified silver nanoparticles to quantify KIM-1 in the kidney by urine analysis to achieve early non-invasive diagnosis of acute kidney injury, with low toxicity and good biocompatibility.

### TECHNICAL FIELD

The invention relates to the technical field of medical materials, especially to KIM-1 active targeting gold nanoparticles (Au-NPs), a preparation method, and an application thereof.

### BACKGROUND

Acute kidney injury (AKI) is a common clinical syndrome, and its early diagnosis is crucial for improving patient prognosis. Currently, the clinical detection of AKI primarily relies on renal injury biomarkers. However, some commonly used renal function indicators have certain limitations. For instance, neutrophil gelatinase-associated lipocalin (NGAL) lacks specificity, as it is expressed not only in the kidney but also in multiple organs such as the lungs and small intestine, and can be non-specifically elevated in systemic inflammation (e.g., sepsis), leading to a tendency for false-positive results. Another biomarker, N-acetyl-β-D-glucosaminidase (NAG), although indicative of tubular injury, is susceptible to interference from urine pH and inhibitory activity of metal ions, resulting in poor stability. Even the most widely used renal function index, serum creatinine (sCr), suffers from low sensitivity and poor specificity. It typically shows a significant increase only 48-72 hours after renal injury and is influenced by factors such as age, gender, and muscle mass, making early warning unattainable.

Kidney injury molecule-1 (KIM-1) exhibits a high degree of renal tubular specificity. It is hardly expressed in healthy kidneys but is significantly upregulated within 12 hours after proximal tubular epithelial cell damage (e.g., due to ischemia or nephrotoxic injury) and remains highly expressed throughout the entire process from injury to repair. Compared with NGAL, KIM-1 offers distinct advantages, including renal tubular specificity, ultra-early warning capability, high stability, and strong prognostic relevance, it has thus emerged as the optimal biomarker for renal injury at present, effectively avoiding false-positive interference caused by systemic inflammation.

KIM-1, which is highly expressed in damaged kidneys, is shed over time and cleared into the urine. Therefore, urinary KIM-1 levels are currently measured in clinical practice to reflect renal function status. However, this approach still faces issues such as time lag. Hence, accurate and non-invasive assessment of KIM-1 expression levels in the kidney is crucial for the early detection of kidney disease. Currently, clinical detection of KIM-1 relies on enzyme-linked immunosorbent assay (ELISA) of urine samples, but this method has several limitations: first, the detection process is time-consuming (>6 hours); second, antibodies are expensive and susceptible to interference from biological matrices; third, it only provides in vitro and static concentration information, unable to localize KIM-1 distribution in the kidney or evaluate its renal content and injury progression in real time; fourth, cross-reactivity due to nonspecific antibody binding to similar biomolecular fragments often affects detection accuracy.

Computed tomography (CT) is one of the most commonly used clinical imaging modalities. Renal CT contrast agents hold promise for non-invasive evaluation of renal KIM-1 levels. Currently, clinical CT contrast agents are mainly iodine-based small-molecule compounds (e.g., iohexol, diatrizoate). These agents enhance imaging by improving tissue density contrast but have three key drawbacks: first, they lack specificity and cannot reflect molecular-level information in the kidney, such as KIM-1 expression; second, they pose nephrotoxicity risks. Over 90% of iodine-based contrast agents are excreted through the kidneys. High concentrations can induce renal vasoconstriction and tubular epithelial cell damage, leading to contrast-induced nephropathy (CIN). This risk is particularly elevated in patients with renal insufficiency, significantly increasing the likelihood of acute kidney injury. Third, iodine-based compounds have a short circulation time in the body and are rapidly cleared (usually within 24 hours), resulting in a narrow imaging time window and making continuous dynamic monitoring difficult.

Therefore, developing a method capable of actively targeting KIM-1 to achieve early, non-invasive, and in vivo diagnosis of AKI is of significant importance and value.

### SUMMARY

The purpose of the invention is to provide KIM-1 active targeting gold nanoparticles, a preparation method, and an application thereof to solve the problems in the above background technology.

In order to achieve the above purpose, the invention provides a preparation method for KIM-1 active targeting gold nanoparticles, including the following steps:
S1, synthesis of glutathione-AuNPs (GS-AuNPs)
(1) dissolving glutathione (GSH) in deionized water and then adding tetrachloroauric acid, heating a mixture to 95° C., and stirring to obtain a reaction solution;
(2) cooling to room temperature, then adding saturated sodium chloride to the above reaction solution, fully stirring, and then adding anhydrous ethanol, fully shaking and centrifuging, collecting the precipitate as GS-AuNPs;
S2, synthesis of gold nanoparticles S-Au-Agent with KIM-1 active targeting
(1) dissolving GS-AuNPs in a PBS buffer solution, adjusting pH to 4.5-5.5 by adding hydrochloric acid solution, and then adding coupling agents EDC and NHS;
(2) adding serine to a reaction system after a vortex operation is performed, and adding a sodium hydroxide aqueous solution to adjust the pH value to 7.8, and then using a vortex mixer for vortex operation;
(3) collecting a reaction liquid after the vortex operation and purifying by ultrafiltration, and collecting an upper liquid to obtain a target product S-Au-Agent.

In some embodiments, in S1 (1), a mass volume ratio of glutathione, tetrachloroauric acid, and deionized water in the mixed system is 36.87 mg:59.07 mg:50 mL.

In some embodiments, in S1 (2), a volume ratio of saturated sodium chloride to anhydrous ethanol is 5 mL:55 mL.

In some embodiments, in S2 (1 ), a mass volume ratio of GS-AuNPs to PBS is 10 mg:5 mL, and a pH of PBS buffer solution is 7.4; a mass ratio of EDC to NHS is 50 mg:60 mg; a molar concentration of hydrochloric acid solution is 1 M.

In some embodiments, in S2 (2 ), a mass of serine is 100 mg; a molar concentration of sodium hydroxide aqueous solution is 10 M.

The invention also provides KIM-1 active targeting gold nanoparticles prepared by the above preparation method.

The invention also provides the gold nanoparticles as a CT contrast agent to detect KIM-1 in a kidney by CT imaging.

The invention also provides the above gold nanoparticles and phosphatidylcholine-modified silver nanoparticles to quantitatively analyze a content of KIM-1 in the kidney by urine analysis.

In some embodiments, a preparation of the phosphatidylcholine-modified silver nanoparticles includes the following steps:
S1, synthesis of GS-AgNPs
adding a mixed solution of AgNO₃ and GSH to deionized water, after stirring to turbidity, adding NaOH solution to adjust a pH value to 4.9-5.1, after constant stirring to clarification, adding NaBH₄, and stirring for a reaction at room temperature, and collecting a reaction solution and filtering to obtain GS-AgNPs;
S2, synthesis of phosphatidylcholine ligand NH₂-MPC
dissolving 2-methacryloyloxyethyl phosphorylcholine in methanol and deoxygenating with nitrogen, and then mixing with the methanol solution of cysteamine hydrochloride, adding triethylamine to catalyze the reaction, after the reaction is completed, removing an excess solvent by rotary evaporation, and dissolving and purifying a product by a mixed solution of dichloromethane and ether, finally, freeze-drying to obtain NH₂-MPC;
S3, synthesis of phosphatidylcholine modified silver nanoparticles PC-AgNPs
dispersing the GS-AgNPs in 1×PBS buffer solution, and activating a carboxyl group by mixing with coupling agents EDC and NHS dissolved in 1×PBS buffer solution, respectively; then, adding NH₂-MPC dissolved in 1×PBS buffer solution for mixing, and adjusting the pH to 7.8 with a sodium hydroxide aqueous solution and stirring for reaction at room temperature, coupling the phosphatidylcholine ligand with the nanoparticles; after the reaction is completed, purifying the PC-AgNPs by ultrafiltration and lyophilizing to obtain PC-AgNPs.

In some embodiments, a mass ratio of AgNO₃, GSH and NaBH₄ in S1 is 34 mg:61.4 mg:38 mg;
in S 2, a mass volume ratio of 2-methacryloyloxyethyl phosphorylcholine and methanol is 100 mg:10 mL; a mass volume ratio of cysteamine hydrochloride to methanol is 28.7 mg:1 mL;
in S3, the mass volume ratio of GS-AgNPs to 1×PBS buffer solution is 2 mg:1 mL, and the pH of 1×PBS buffer solution is 4.5;
the mass volume ratios of EDC, NHS and 1×PBS buffer solution are 7 mg:1 mL and 4 mg:1 mL, respectively; the pH of 1×PBS buffer solution is 4.5;
the mass volume ratio of NH 2-MPC to 1×PBS buffer solution is 3 mg: 1 mL, and the pH of 1×PBS buffer solution is 7.8.

Therefore, the KIM-1 active targeting gold nanoparticle and the preparation method and the application thereof provided by the invention have the following beneficial effects:

### (1) High Imaging Performance and Targeting of S—Au-Agent

The atomic number of gold is 79, while that of iodine is 53, the X-ray mass absorption coefficient of gold (5.16 cm²/g) is about 2.7 times that of iodine (1.94 cm²/g). At the same dose, S-Au-Agent can provide clearer CT image contrast, especially for imaging obese mice or deep kidney tissues. Meanwhile, owing to its ultra-small size (core diameter 2.19±0.86 nm, hydration kinetic diameter 3.97±1.31 nm), S-Au-Agent exhibits a longer clearance half-life in vivo compared with iodine-based contrast agents, which extends the imaging window of renal CT angiography to more than 30 minutes and facilitates continuous dynamic monitoring. Additionally, the active targeting ability of S-Au-Agent toward KIM-1 is conferred by serine as a ligand, enabling specific accumulation in the renal injury region, particularly in the cortical proximal tubules, and enhancing microscopic contrast at the injury site, this allows for early diagnosis of renal injury and non-invasive observation of KIM-1 distribution in the kidney.

### (2) Non-Invasiveness and Accuracy of Urine Element Ratio Analysis

The invention designs and synthesizes another inorganic agent, silver nanoparticles (PC-AgNPs), which function as an internal reference. Their inorganic interaction characteristics ensure that their renal metabolism is not influenced by KIM-1 levels, thereby effectively offsetting interference from individual physiological variations, such as fluctuations in glomerular filtration rate and differences in metabolic rate, on test results. Leveraging the active targeting ability of S—Au-Agent toward renal KIM-1 and the stable internal reference capability of PC-AgNPs, the contents and ratio of KIM-1 and PC-AgNPs in urine can be determined via elemental mass spectrometry. The ratio of silver to gold in urine increases with rising renal KIM-1 content, thereby enabling quantitative assessment of renal KIM-1 levels through urine analysis and achieving non-invasive quantitative evaluation of KIM-1 in the kidney. Compared with conventional urine KIM-1 detection, which shows a significant difference only 48 hours after renal ischemia-reperfusion, this method detects acute kidney injury at least 42 hours earlier. It avoids the time lag and heterogeneity associated with traditional urine testing, is easy to perform, does not require complex tissue sampling, and can be completed using only urine samples.

### (3) Low Toxicity and Clinical Applicability

Both S—Au-Agent and PC-AgNPs are ultra-small nanoparticles, surface-modified with serine and phosphatidylcholine, respectively. They demonstrate good biocompatibility and can be cleared through normal renal metabolism, preventing accumulation and toxicity in the body. Compared with iodine contrast agents, S—Au-Agent achieves favorable imaging results without high-concentration injection, significantly reducing the risk of contrast-induced nephropathy (CIN), particularly beneficial for high-risk groups with renal insufficiency.

The technical scheme of the invention is further elaborated below through drawings and implementation examples.

### BRIEF DESCRIPTION OF THE DRAWINGS

FIG. 1 is a synthesis roadmap of ultra-small gold nanoparticles with serine as ligand in the embodiment of the invention;

FIG. 2 is a core diameter diagram of the S—Au-Agent obtained in the embodiment of the invention;

FIG. 3 is a hydrodynamic diameter diagram of the S—Au-Agent obtained in the embodiment of the invention;

FIG. 4 is an excitation/emission spectrum and ultraviolet-visible absorption spectrum of the S—Au-Agent obtained in the embodiment of the invention;

FIG. 5 is an agarose gel electrophoresis diagram of S—Au-Agent obtained in the embodiment of the invention;

FIG. 6 is an in vitro CT signal intensity diagram of S—Au-Agent and iodine contrast agent obtained in the embodiment of the invention. Among them, A is a CT imaging diagram of S—Au-Agent and iopromide solution at different concentrations; B is a linear relationship between the concentration of the two contrast agents and the CT enhancement, and the slope indicates the efficiency of the contrast agent with the increase of the concentration;

FIG. 7 is a kidney imaging effect of S—Au-Agent in the sham operation group mice;

FIG. 8 is a kidney imaging effect diagram of S—Au-Agent in ischemia-reperfusion mice prepared by the embodiment of the invention;

FIG. 9 is a flow chart of the urinalysis of S—Au-Agent and PC-AgNPs obtained by the embodiment of the invention to quantitatively evaluate KIM-1 in the kidney and detect acute kidney injury;

FIG. 10 is a content of KIM-1 in the kidney and urine of mice after different time of ischemia-reperfusion;

FIG. 11 shows immunohistochemical maps of the kidneys of mice in the sham operation group and the 6 h ischemia-reperfusion group; among them, A is the immunohistochemical map of the sham operation group and the 6 h ischemia-reperfusion renal injury group; B is the quantitative analysis of the positive region of KIM-1 in FIG. A;

FIG. 12 is a content of gold and silver in the kidney and the ratio of the two elements, where A is the content of silver in the kidney; B is the content of gold in the kidney; C is the ratio of silver and gold in the kidney;

FIG. 13 is a content of gold and silver in urine and the ratio of the two elements, where A is the content of silver in urine; B is the content of gold in urine; C is the ratio of silver and gold in urine;

FIG. 14 is a linear fitting diagram of the correlation between the ratio of silver and gold in kidney and urine and the content of KIM-1 in kidney; among them, A is a linear fitting of the content of KIM-1 in kidney and the ratio of silver and gold in kidney; B is the linear fitting of the ratio of KIM-1 content in kidney to the content of silver and gold in urine.

### DETAILED DESCRIPTION OF THE EMBODIMENTS

The following is a further explanation of the technical scheme of the invention through drawings and implementation examples. It should be understood that these embodiments are only used to illustrate the invention and not to limit the scope of the invention. Any other changes, modifications, substitutions, combinations, and simplifications that do not violate the spiritual essence and principle of the invention should be equivalent replacement methods and are included in the protection range of the invention. In addition, it should be understood that after reading the content of the invention, technicians in this field can make various changes or modifications to the invention. These equivalent forms also fall within the scope of the claims attached to the application, which belong to the scope of protection of the invention.

The reference to “embodiment” in this article means that a particular feature, structure, or characteristic described in conjunction with an embodiment may be included in at least one embodiment of this application. The word “embodiment” appearing in each position of the instruction does not necessarily refer to the same embodiment, nor does it specifically limit its independence or relevance to other embodiments. In principle, in this application, as long as there is no technical contradiction or conflict, the technical features mentioned in each embodiment can be combined in any way to form the corresponding technical scheme that can be implemented.

Unless otherwise defined, the technical terms used herein shall have the same meaning as those normally understood by the technical staff in the technical field to which the application relates; the use of relevant terms in this article is intended only to describe specific embodiments, not to limit the application.

If there is no special description in the invention, the reagents, instruments, equipment, and performance test methods used are all reagents, instruments, equipment, and methods commonly used by technicians in this field.

### Embodiment

This embodiment provides a preparation method for KIM-1 active targeting gold nanoparticles, including the following steps:
(1) 36.87 mg of glutathione is dissolved in 50 mL of water in a three-necked flask, and 59.07 mg of tetrachloroauric acid is added to the reaction system, the above mixed system is heated at 95° C. and stirred at 420 rpm for 40 min.
(2) After the reaction is cooled to room temperature, 5 mL of saturated sodium chloride is added to the reaction solution, and 55 mL of anhydrous ethanol is added after full stirring. After being fully shaken, it is placed in a 50 mL centrifuge tube at 4200 rpm for 10 min, and the precipitate is collected to obtain GS-AuNPs.
(3) 10 mg of GS-AuNPs is dissolved in 5 mL PBS (pH =7.4) in a penicillin bottle, and the pH is adjusted to 4.5-5.5 with 1M hydrochloric acid, then 50 mg of EDC and 60 mg of NHS are added to the system, and vortexed at 600 rpm for 2 h.
(4) 100 mg of serine is added to the reaction system after the vortex is completed, and 40 μL of sodium hydroxide aqueous solution (10 M) is added to adjust the pH value to 7.8, after that, the vortex mixer is used to vortex at 600 rpm for 3 h.
(5) The reaction solution is collected and filtered using a 3000 Da ultracentrifugal tube to remove unreacted EDC, NHS, and serine. After 5 times of filtration, the upper liquid is collected to obtain S—Au-Agent.

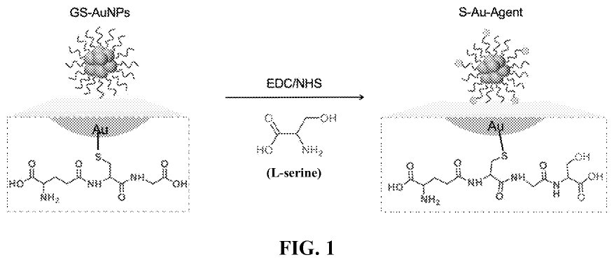

Steps (3)-(5) are the synthesis of ultra-small gold nanoparticles with serine as ligand, and the schematic diagram of the synthesis route is shown in FIG. 1.

The basic physicochemical properties of the target product S—Au-Agent prepared by the embodiment are characterized.

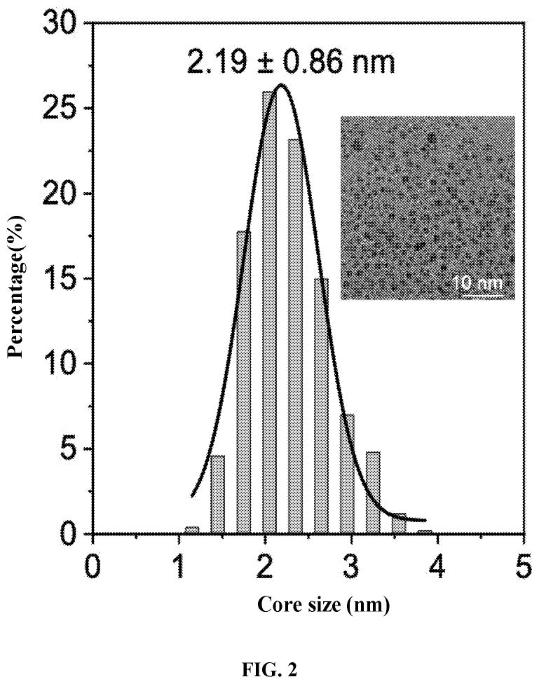

The core size and morphology of the nanoparticles are characterized by transmission electron microscopy (TEM), and the results are shown in FIG. 2. It can be seen from FIG. 2 that the core diameter of S—Au-Agent is 2.19±0.86 nm, which is spherical and conforms to the size range of ultra-small gold nanoparticles.

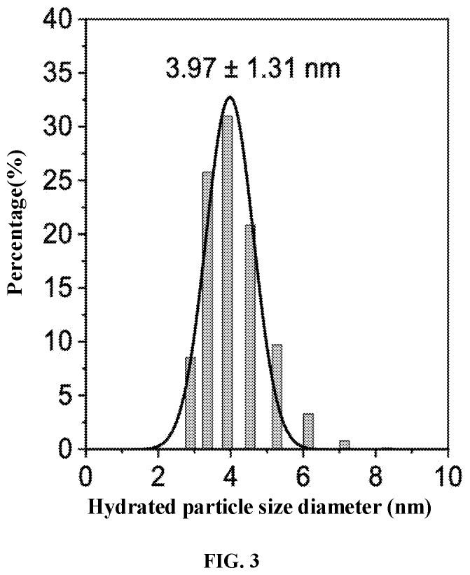

The hydrodynamic diameter of the nanoparticles is characterized by dynamic light scattering (DLS), and the results are shown in FIG. 3. It can be seen from FIG. 3 that the hydrodynamic diameter (hydrated particle size) of S—Au-Agent is 3.97±1.31 nm, which is in line with the size range of ultra-small gold nanoparticles.

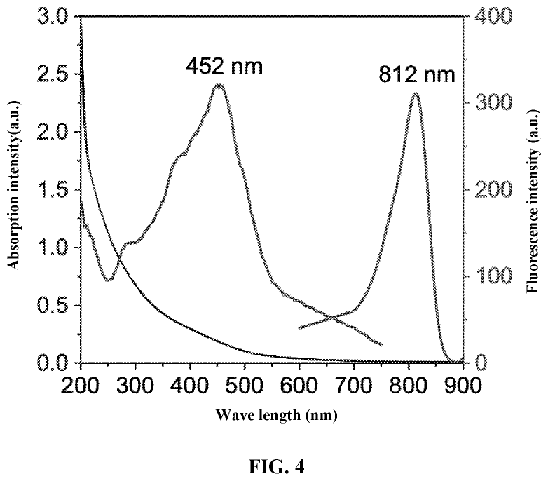

The characteristic absorption of nanoparticles is characterized by an ultraviolet-visible spectrophotometer (UV-Vis), and the fluorescence properties of nanoparticles are characterized by a fluorescence spectrometer (FL). The results are shown in FIG. 4. It can be seen from FIG. 4 that S—Au-Agent has fluorescence properties, the optimal excitation wavelength is 452 nm, and the maximum emission wavelength is 812 nm. In the range of 400-500 nm, there is no obvious UV-Vis absorption of nanoparticles, which proves that there is no surface plasmon resonance and belongs to ultra-small size nanoparticles.

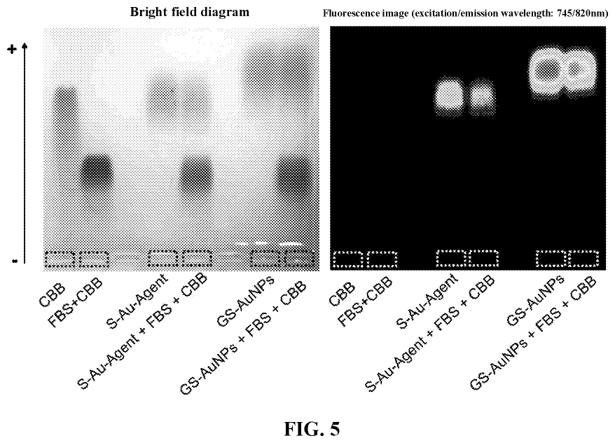

The S—Au-Agent and GS-AuNPs are compared by agarose gel electrophoresis, and the results are shown in FIG. 5. It can be seen from FIG. 5 that the synthesis of S—Au-Agent can be proved by agarose gel electrophoresis experiment, and its swimming speed in electrophoresis is slower than that of GS-AuNPs, which proves that serine is successfully connected to the surface of gold nanoparticles.

Application example 1, the serine-modified ultrasmall gold nanoparticle S—Au-Agent prepared by the embodiment is used as a CT contrast agent for CT imaging to detect KIM-1 in the kidney.

### 1, In Vitro CT Imaging Performance Evaluation of S—Au-Agent

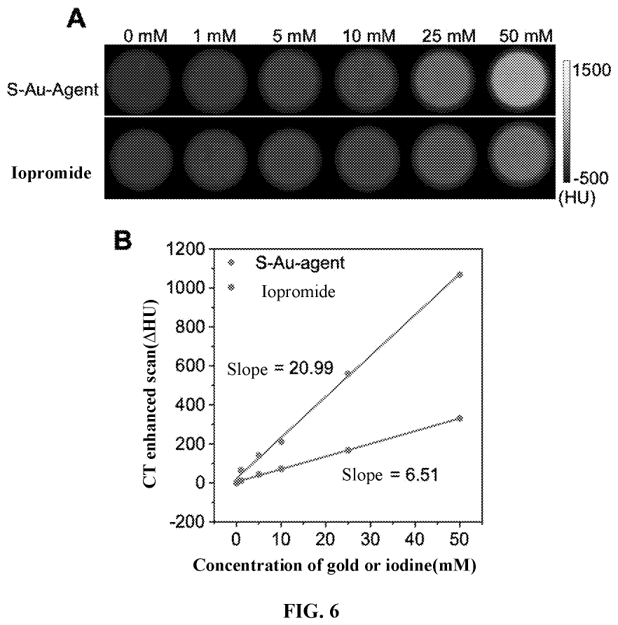

The CT imaging ability of S—Au-Agent is characterized in vitro using small animal Micro-CT. Specifically, S—Au-Agent is dissolved in PBS to prepare a solution of 100 mg/mL, and 100 μL of the solution is added to an independent 96-well plate, the iodine element with the same molar number as the gold element and PBS are used as controls. The signal value difference between the iodine contrast agent (iopromide) and the gold nanoparticles under the same mass of the imaging elements (gold and iodine) is compared to clarify the CT-enhanced imaging ability of the gold nanoparticles. A CT scan is performed using a small animal Micro-CT with a scanning parameter of 70 kV and a scanning time of 2 min, pre-scanning is performed before the injection of nanoparticles to obtain an unenhanced kidney region image. After the injection of nanoparticles, 15 consecutive scans are performed for a total of 30 minutes to obtain a kidney region image enhanced by gold nanoparticles. The results are shown in FIG. 6. From FIG. 6, it can be seen that S—Au-Agent has a signal intensity of 3.22 times higher than that of the iodine contrast agent in the case of equal mass of imaging elements (equal molar of gold and iodine).

### 2, In Vivo CT Imaging Performance Evaluation of S—Au-Agent

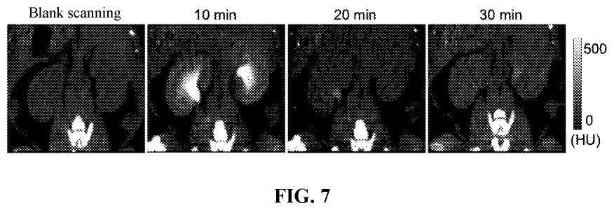

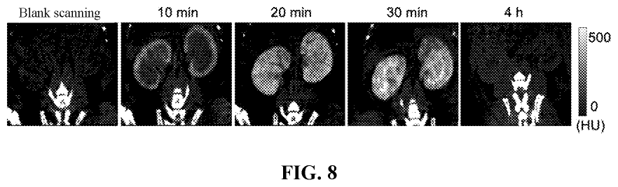

The mice model of ischemia-reperfusion (IRI) renal injury with increased KIM-1 is constructed. The openings of the bilateral kidneys on the back of the mice are clamped with an artery clip for 30 min, and the artery clip is loosened to restore blood flow for reperfusion. After 6 h, renal CT imaging is performed. A sham operation group is established for comparison, the skin of the corresponding area on the back of the mice is sutured in the sham operation group. The S—Au-Agent is injected intravenously into mice with a sham operation group and IRI renal injury at a dose of 1.8 mg/mouse. The micro-CT of the above small animals is continuously scanned for 2 min/time, and the scanning is performed 15 times. The differences between kidney imaging under normal and disease conditions are compared, and the signal intensity of the kidney region is analyzed using software. The results are shown in FIG. 7 and FIG. 8. It can be seen from FIG. 7 that when S—Au-Agent (mass of Au element) is injected into the sham operation group, S—Au-Agent is rapidly transported in the mice and clearly lights up the renal pelvis of the mice. This is because KIM-1 is almost not expressed in the kidney of the sham operation group, so S—Au-Agent is rapidly cleared in the kidney and accumulated significantly in the renal pelvis. As shown in FIG. 8, it can be seen that compared with the sham operation group, the S—Au-Agent of the IRI group mice shows obvious cortical accumulation in the renal cortex area in the early stage (the first 10 minutes) after injection, and then the S—Au-Agent is gradually transported from the cortex to the whole kidney after 20 min and 30 min after injection, and finally cleared after 4 h. The early enrichment of this cortical region is highly consistent with the spatial distribution of KIM-1 high expression in the proximal tubules (located in the cortex) of the kidney, demonstrating the excellent KIM-1 targeting ability of S—Au-Agent. Based on this, the up-regulation of KIM-1 can be detected by non-invasive imaging of S—Au-Agent, as well as the early diagnosis of renal injury, so as to realize the in vivo, non-invasive, and dynamic evaluation of KIM-1 expression level.

### 3. Comparison and Evaluation of S—Au-Agent Non-Invasive CT Imaging Mode and Kidney KIM-1 Content

(1) After CT imaging, the mice are euthanized, and the kidneys of the mice are collected, the content of KIM-1 in the kidneys of the mice is determined by enzyme-linked immunosorbent assay, and the spatial distribution of KIM-1 in the kidneys is quantified and stained by immunohistochemistry, the spatial distribution of S—Au-Agent in the kidney during CT imaging and the correlation of KIM-1 distribution in immunohistochemistry are compared, the early diagnosis and microstructure localization of kidney disease is achieved.
(2) Meanwhile, the serum of mice is collected to determine the renal function indexes, such as serum creatinine and urea nitrogen, so that it can be compared with the detection method of CT imaging to determine whether early renal injury detection could be carried out by non-invasive imaging.
(3) After the kidneys are removed, 4% paraformaldehyde is used for tissue fixation, paraffin-embedded sections are stained with hematoxylin and eosin (H&E), and the degree of renal tubular injury is analyzed; ten high-power field of view are randomly selected for each kidney section, and scored according to the following criteria: obvious expansion of renal tubules, flat cell is scored for 1 point; brush edge damageis scored 1 point, shedding is scored 2 points; the tube type is 2 points, and the shedding and necrotic cells (not forming tube type or cell debris) in the renal tubular lumen are 1 point; the highest score of each field of vision is 4 points, and the lowest score is 0 points. Similarly, CT detection is used to compare with pathological analysis, and the pathological changes caused by renal injury are fed back through CT imaging technology.

Application example 2: Quantitative analysis of KIM-1 content in the kidney and early detection of renal injury by urine analysis using S—Au-Agent combined with phosphatidylcholine-modified silver nanoparticles prepared by the embodiment.

The phosphatidylcholine-modified silver nanoparticles have the function of metabolic internal reference, the preparation method includes the following steps:
(1) Synthesis of GS-AgNPs: 100 mL of deionized water is added to a three-neck flask, and 34 mg of AgNO₃ and 61.4 mg of GSH solution are added at 900 rpm, then 60 μL of 5M NaOH solution is added to adjust the pH value of the solution to 4.9-5.1, and the solution is clarified by constant stirring. At this time, 38 mg of NaBH₄ is quickly added to a three-neck flask, and the reaction is stirred violently at 900 rpm at room temperature for 12 h, the reaction solution is collected and purified overnight with a 1500 Da dialysis bag to synthesize GS-AgNPs.
(2) Synthesis of phosphatidylcholine ligand NH₂-MPC: 100 mg of 2-methacryloyloxyethyl phosphorylcholine is dissolved in 10 mL of methanol, and nitrogen is passed for 15 min. 28.7 mg cystine hydrochloride is dissolved in 1 mL of methanol solution. After fully dissolving, the above two solutions are mixed, and then 10 uL of triethylamine is added. The reaction is stirred at 400 rpm for 4 hours at room temperature. After the reaction, the excess solvent is removed by a rotary evaporator, and then the product is re-dissolved with a mixed solution of 10 mL of dichloromethane and ether. After standing for 30 min, the supernatant is removed, and the product is collected. Finally, the excess solvent is removed by rotary evaporation, and 1 mL of distilled water is added to fully dissolve the product. After lyophilization, the phosphatidylcholine ligand NH₂-MPC is obtained.
(3) Synthesis of phosphatidylcholine modified silver nanoparticles PC-AgNPs: 2 mg of GS-AgNPs are fully dissolved in 1×PBS buffer solution (pH=4.5). 7 mg of 1-ethyl-(3-dimethylaminopropyl) carbodiimide hydrochloride and 4 mg of N-hydroxysuccinimide are dissolved in 1 mL of 1×PBS buffer solution (pH=4.5), respectively. The above system is mixed and stirred at room temperature for 2 h. Then, 3 mg of NH₂-MPC is fully dissolved in 1 mL of 1×PBS buffer solution (pH=7.8). Subsequently, the above four solutions are mixed, and 40 μL of sodium hydroxide aqueous solution (10 M) is added to adjust the pH value to 7.8 and stirred at room temperature for 6 h. After the reaction, the reaction system is transferred to a 3 kDa ultracentrifugal tube for ultrafiltration purification, and PC-AgNPs are obtained after lyophilization.

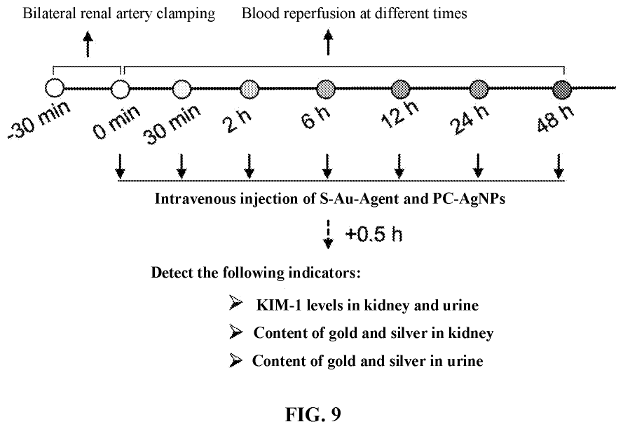

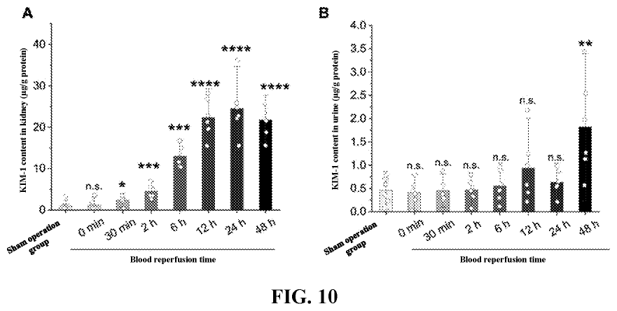

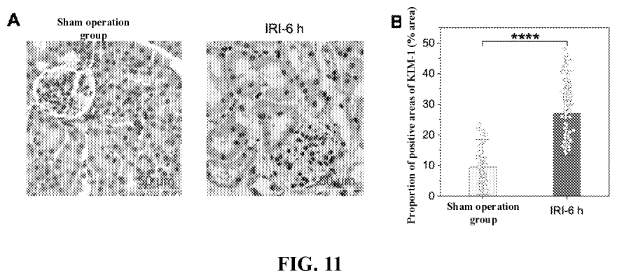

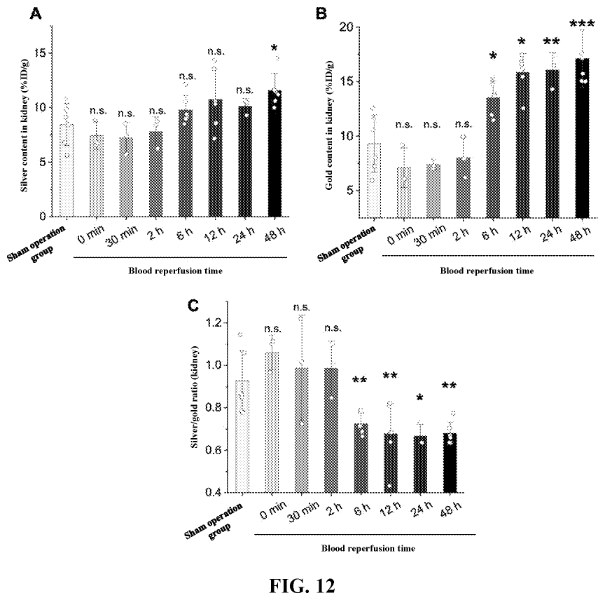

The flow chart of the urine test is shown in FIG. 9. The mice model and sham operation group at different time points (0, 30 min, 2, 6, 12, 24, 48 h) after IRI are constructed as described above. Each group of mice is injected with S—Au-Agent and PC-AgNPs (20 μg each) through the tail vein at the same time. After 30 minutes of injection, the urine of the mice is collected, and then the mice are sacrificed and the kidneys are removed, a part of kidney tissue is used to quantify KIM-1 content by ELISA and immunohistochemistry, and the results are shown in FIG. 10 and FIG. 11. Another part of the kidney tissue and all urine samples are nitrated by nitric acid, and the contents of gold (Au) and silver (Ag) are detected by mass spectrometry. The results are shown in FIG. 12. According to the principle of element ratio method to reflect the level of KIM-1 in kidney, when KIM-1 is highly expressed in kidney, the kidney accumulation of S—Au-Agent increases and the content of S—Au-Agent in urine decreases, but the silver nanoparticles without any specific targeting will not be changed by the up-regulation of KIM-1, so the level of KIM-1 in kidney can be reflected by analyzing the Au/Ag ratio in urine. Meanwhile, the advantage of the ratio method is that it can remove the interference in the organism, and the result has higher accuracy.

It can be seen from FIG. 10 that there is a lag in the detection of KIM-1 in urine, and there is a significant difference between urine and normal mouse urine after 48 h of reperfusion. The content of KIM-1 in the kidney of mice increases significantly after 30 min of reperfusion, but it is still in the normal range. Therefore, the early stage with an obvious increase of KIM-1, namely 6 h of reperfusion, is selected as the experimental research object for CT imaging detection.

It can be seen from FIG. 11 that the kidneys of mice in the sham operation group and the kidneys of mice with 6 h ischemia-reperfusion are selected for immunohistochemical staining. It can be seen from the histochemical results that KIM-1 is highly expressed in the proximal tubules due to renal injury (FIG. 11A), resulting in a 2.72-fold higher KIM-1 positive area in the cortex than in the sham operation group (FIG. 11B).

It can be seen from FIG. 12 that the accumulation of silver in the kidney after 30 min of injection of the two nanoparticles is reflected. It can be seen that with the prolongation of renal reperfusion time, the accumulation of silver does not change significantly until the experimental group at 48 h of reperfusion is slightly increased. This is because the damage has caused the decrease of glomerular filtration rate in the organic lesions of the kidney (FIG. 12 A), while the accumulation of gold has increased at 6 h of reperfusion (FIG. 12 B), which is consistent with the previous detection of KIM-1 content. This is because the increase in KIM-1 increases the ability of the proximal renal tubule to take up S—Au-Agent. Similarly, the ratio of the two elements in the kidney also reflects the same trend (FIG. 12B).

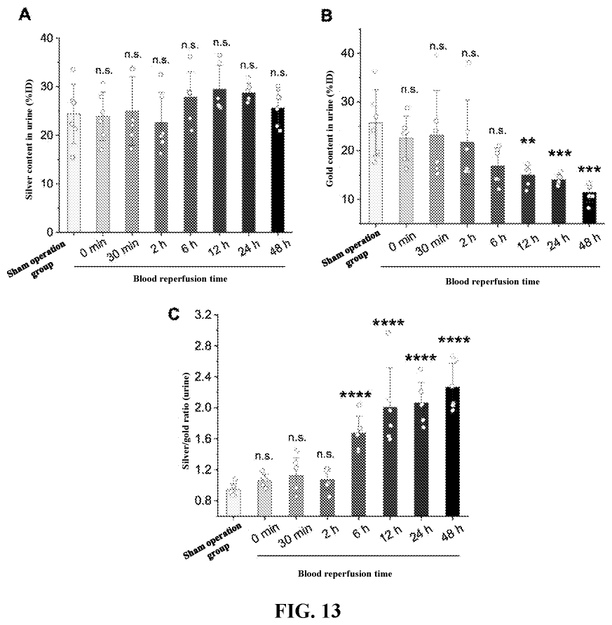

The removal efficiency of gold and silver nanoparticles in urine and the ratio of the two elements are further analyzed. The results are shown in FIG. 13. Because of the increased accumulation of kidneys, the clearance efficiency in urine is reduced, and it is difficult for silver nanoparticles to feed back this trend, which proves the stability of the internal reference (FIG. 13 A). The gold nanoparticles can feedback this phenomenon after 12 h of reperfusion, and the removal efficiency will decrease with the increase of KIM-1 content in the kidney (FIG. 13 B). By calculating the ratio of each reperfusion time period using the ratio method, it can be observed that the occurrence of acute kidney injury is found at least 6 hours earlier by this method, and the heterogeneity of the urine test is avoided (FIG. 13 C).

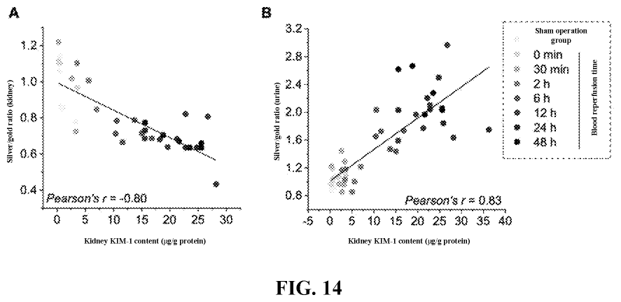

The correlation curve between the ratio of silver and gold in the kidney and urine of all experimental mice and the content of KIM-1 in the kidney is shown in FIG. 14. The content of KIM-1 in the kidney is set as the horizontal axis, and the ratio of silver and gold in the urine and kidney is set as the vertical axis for linear fitting. It can be clearly observed that the ratio of silver and gold in the kidney is negatively correlated with the content of KIM-1 in the kidney, which is due to the increase in the relative accumulation of gold in the kidney. On the contrary, the ratio of silver to gold in urine is negatively correlated with the content of KIM-1 in the kidney, both of which have a good correlation. This linear relationship can be used to quantitatively evaluate the content of KIM-1 in the kidney and realize the early detection of renal injury through urine analysis.

Finally, it should be explained that the above embodiments are only used to explain the technical scheme of the invention rather than restrict it. Although the invention is described in detail with reference to the better embodiment, the ordinary technical personnel in this field should understand that they can still modify or replace the technical scheme of the invention, and these modifications or equivalent substitutions cannot make the modified technical scheme out of the spirit and scope of the technical scheme of the invention.

What is claimed is:

1. A preparation method for KIM-1 active targeting gold nanoparticles, comprising the following steps:
S1, synthesis of glutathione-gold nanoparticles (GS-AuNPs)
(1) dissolving glutathione in deionized water and then adding tetrachloroauric acid, heating a mixture to 95° C., and stirring to obtain a reaction solution;
(2) cooling to room temperature, then adding saturated sodium chloride to the above reaction solution, fully stirring and then adding anhydrous ethanol, fully shaking and centrifuging, collecting a precipitate as GS-AuNPs;
S2, synthesis of gold nanoparticles S—Au-Agent with KIM-1 active targeting (1) dissolving GS-AuNPs in a PBS buffer solution, and adjusting pH to 4.5-5.5 by adding hydrochloric acid solution, and then adding coupling agents EDC and NHS, and mixing with a vortex mixer;
(2) adding serine to a reaction system after vortex mixing is performed, and adding a sodium hydroxide aqueous solution to adjust the pH value to 7.8, and then mixing with a vortex mixer;
(3) collecting a reaction liquid after vortex mixing and purifying by ultrafiltration, and collecting a supernatant to obtain a target product S—Au-Agent;
wherein the gold nanoparticles prepared by (3) are used as a CT contrast agent to detect KIM-1 in a kidney by CT imaging; and
wherein the gold nanoparticles prepared by (3) and phosphatidylcholine-modified silver nanoparticles are used to quantitatively analyze a content of KIM-1 in the kidney by a urinalysis.

2. The preparation method for KIM-1 active targeting gold nanoparticles according to claim 1, wherein in S1 (1), a mass volume ratio of glutathione, tetrachloroauric acid, and deionized water in the mixed system is 36.87 mg:59.07 mg:50 mL.

3. The preparation method for KIM-1 active targeting gold nanoparticles according to claim 1, wherein in S1 (2), a volume ratio of saturated sodium chloride to anhydrous ethanol is 5 mL:55 mL.

4. The preparation method for KIM-1 active targeting gold nanoparticles according to claim 1, wherein in S2 (1), a mass volume ratio of GS-AuNPs to PBS is 10 mg:5 mL, and a pH of PBS buffer solution is 7.4; a mass ratio of EDC to NHS is 50 mg:60 mg; a molar concentration of hydrochloric acid solution is 1 M.

5. The preparation method for KIM-1 active targeting gold nanoparticles according to claim 1, wherein in S2 (2), a mass of serine is 100 mg; a molar concentration of sodium hydroxide aqueous solution is 10 M.

6. The preparation method for KIM-1 active targeting gold nanoparticles according to claim 1, wherein a preparation of the phosphatidylcholine-modified silver nanoparticles comprises the following steps:
S1, synthesis of GS-AgNPs:
adding a mixed solution of AgNO₃ and GSH to deionized water, after stirring to turbidity, adding NaOH solution to adjust a pH value to 4.9-5.1, after constant stirring to clarification, adding NaBH₄, and stirring for a reaction at room temperature, and collecting a reaction solution and filtering to obtain GS-AgNPs;
S2, synthesis of phosphatidylcholine ligand NH₂-MPC:
dissolving 2-methacryloyloxyethyl phosphorylcholine in methanol and deoxygenating with nitrogen, and then mixing with a methanol solution of cysteamine hydrochloride, adding triethylamine to catalyze the reaction, after the reaction is completed, removing excess solvent by rotary evaporation, and dissolving and purifying a product by a mixed solution of dichloromethane and ether, and finally, freeze-drying to obtain NH2-MPC;
S3, synthesis of phosphatidylcholine modified silver nanoparticles PC-AgNPs:
dispersing the GS-AgNPs in 1×PBS buffer solution, and activating a carboxyl group by mixing with coupling agents EDC and NHS dissolved in 1×PBS buffer solution, respectively; then, adding NH₂-MPC dissolved in 1×PBS buffer solution for mixing, and adjusting the pH to 7.8 with a sodium hydroxide aqueous solution and stirring for reaction at room temperature, thus coupling the phosphatidylcholine ligand with the nanoparticles; after the reaction is completed, purifying the PC-AgNPs by ultrafiltration and lyophilizing to obtain PC-AgNPs.

7. The preparation method for KIM-1 active targeting gold nanoparticles according to claim 6, wherein a mass ratio of AgNO₃, GSH, and NaBH₄ in S1 is 34 mg:61.4 mg:38 mg;
in S2, a mass volume ratio of 2-methacryloyloxyethyl phosphorylcholine and methanol is 100 mg:10 mL; a mass volume ratio of cysteamine hydrochloride to methanol is 28.7 mg:1 mL;
in S3, the mass volume ratio of GS-AgNPs to 1×PBS buffer solution is 2 mg:1 mL, and the pH of 1×PBS buffer solution is 4.5;
wherein the mass volume ratios of EDC, NHS and 1×PBS buffer solution are 7 mg:1 mL and 4 mg:1 mL, respectively; the pH of 1×PBS buffer solution is 4.5;
the mass volume ratio of NH₂-MPC to 1×PBS buffer solution is 3 mg:1 mL, and the pH of 1×PBS buffer solution is 7.8.

8. KIM-1 active targeting gold nanoparticles, wherein the gold nanoparticles are prepared by the preparation method according to claim 1.
